(19) United States
(12) Patent Application Publication
(10) Pub. No.: US 2005/0210536 A1
(43) Pub. Date: Sep. 22, 2005

(54) Modulation of brain pathways and function

(76) Inventors: Edward I. Ginns, Shrewsbury, MA (US); Barry J. Richmond, Bethesda, MD (US)

Correspondence Address: FISH & RICHARDSON PC, P.O. BOX 1022, MINNEAPOLIS, MN, 55440-1022, US

(21) Appl. No.: 11/005,919

(22) Filed: Dec. 6, 2004

Related U.S. Application Data
(60) Provisional application No. 60/527,467, filed on Dec. 5, 2003.

(51) Int. Cl. A01K 67/027

(52) U.S. Cl. 800/12; 800/14

(57) ABSTRACT

Methods and compositions for modulating brain pathways and functions are disclosed.

### CLAIM OF PRIORITY

This application claims priority under 35 USC § 119(e) to U.S. Patent Application Ser. No. 60/527,467, filed on Dec. 5, 2003, the entire contents of which are hereby incorporated by reference.

### TECHNICAL FIELD

This invention relates to the modulation of brain pathways and function, and more particularly to the modulation of cognitive functions.

### BACKGROUND

Animals and humans adjust their behavior based on their prediction of the amount of work, i.e., workload, that it will take to reach a goal or obtain a reward. Often the prediction is made using environmental cues, including visual stimuli. In those circumstances, cues provide associative information about a type of reward expectancy. Disturbances of associations between cues and goals or rewards are prominent in many organic behavioral disorders, such as schizophrenia and cocaine use/abuse (Everitt et al., Brain Res. Reviews, 36:129-138 (2001); Hyman et al., Nature Reviews: Neurosci., 2:695-703 (2001); Volkow et al., J Psychopharmacol., 13:337-345 (1999)).

Classical pharmacological agents can be used in identifying cellular mechanisms underlying performance on certain behavioral tasks, e.g., recognition memory (Tang et al., Proc. Natl. Acad. Sci. USA, 94:2667-2669 (1997)). However, they have certain limitations, especially when studying learning. First, a specific pharmacological agent must be available. Second, the effects of many pharmacological agents are typically short lived; the effective duration of the agent must be carefully evaluated to ensure data collection is contained within that period. Third, because of their short-lived effects, pharmacological approaches are typically limited to performance tests of an already-learned rule, as opposed to active learning itself. Finally, since the tertiary structure of receptors determines their ligand specificity, pharmacological ligands often display binding affinities for multiple subtypes of receptors in the same family.

### SUMMARY

The invention is based, in part, on the discovery that administration of a nucleic acid construct encoding a nucleotide sequence that is complementary to an mRNA of a target gene can be used to inhibit the target gene's function for an extended, yet finite, period of time, and lead to a modulation of cognitive and motor functions.

In one aspect, the invention provides methods of making animal models of neurological disorders, e.g., schizophrenia, cocaine use/abuse, Parkinson's Disease. The methods include administering to a brain region, e.g., the basal ganglia, of an animal, e.g., a non-human primate, a nucleic acid construct including a nucleotide sequence that is complementary to a portion of an mRNA (e.g., a portion of the mRNA that is within the translated or coding region) of a target gene encoding a protein, the activity of which protein is associated with the disorder, in an amount effective to inhibit translation of the mRNA, thereby inducing the disorder in the animal.

In some embodiments, the nucleic acid construct is administered to one side of the brain; the hemisphere to which the nucleic acid construct is not administered can be used a reference. In some embodiments, the target gene encodes a protein within the dopamine pathway. In some embodiments, the target gene encodes tyrosine hydroxylase. The methods can include administering more than one nucleic acid construct, e.g., a plurality of nucleic acid constructs that target the same gene, or a plurality of nucleic acid constructs that target more than one gene.

The invention also includes animal models of neurological disorders produced by methods described herein. In one aspect, the invention includes an animal model of a neurological disorder caused by or associated with decreased expression of a target gene, wherein the animal has inserted into a region of its brain a nucleic acid construct including a nucleotide sequence that is complementary to a portion of an mRNA of the target gene, in an amount effective to inhibit translation of the mRNA. In some embodiments, the region of the brain is only in one hemisphere.

In some embodiments, the target gene is the D2 dopamine receptor and the disorder is selected from the group consisting of Parkinson's Disease, obsessive compulsive disorder, schizophrenia, and drug abuse. In other embodiments, the target gene is dopamine β-hydroxylase and the disorder is affective disorder. Alternatively, the target gene can be neuregulin 1, dystrobrevin binding protein, and disrupted in schizophrenia-1 (DISC-1), and the disorder is schizophrenia.

Typically, the nucleic acid constructs used in the methods described herein include DNA, and can include expression vectors.

In some embodiments, the induction of the disorder in the animal is not permanent, e.g., is reversible after the passage of a period of time.

The invention further provides methods of testing potential therapies for treatment of neurological disorders caused by, or associated with, increased activity of a protein encoded by a gene. The methods include administering to a brain region of a test subject not having the disorder a nucleic acid construct including a nucleotide sequence that is complementary to a portion of an mRNA of the gene, in an amount effective to inhibit translation of the mRNA, thereby inducing the disorder in the test subject; administering a potential therapy; and evaluating an effect of the potential therapy on a clinical parameter of the disorder. An improvement in the clinical parameter indicates that the therapy is effective in treating the neurological disorder.

In some embodiments, the improvement is relative to a control or reference subject, e.g., a subject that has not been administered the therapy. The reference subject can be the same individual as the test subject, prior to induction of the disorder. Where the nucleic acid construct has been administered only to one hemisphere of the brain, the hemisphere to which the nucleic acid construct is not administered can be used as a reference.

The potential therapy can include, e.g., experimental or conventional therapies, e.g., administering a test compound, such as one or more of small organic or inorganic molecules, peptides, polypeptides, nucleic acid sequences, and polysaccharides. A test compound that has been screened by a method described herein and determined to be effective in treating the disorder, e.g., causes an improvement in one or more symptoms or clinical parameters of the disorder, can be considered a candidate therapeutic agent. Candidate therapeutic agents, once screened in a clinical setting such as a clinical trial, can be considered therapeutic agents. Candidate therapeutic agents and therapeutic agents can be optionally optimized and/or derivatized, and formulated with physiologically acceptable excipients to form pharmaceutical compositions.

The potential therapy can be a permanent treatment, e.g., surgery, e.g., surgical removal of tissue from a brain region. The therapy can include administration of a noncompetitive inhibitor, or a viral vector. In some embodiments, the potential therapy can be administration of pharmaceuticals (e.g., L-DOPA and antibiotics), radiotherapy, and/or psychotherapy.

In another aspect, the invention provides methods for selecting candidate nucleic acid constructs for the treatment of neurological disorders. The methods can include administering to a brain region of a subject having the disorder a test nucleic acid construct comprising a nucleotide sequence that is complementary to a portion of a mRNA strand of a pre-selected target gene in an amount effective to inhibit translation of the mRNA in a control subject; evaluating an effect on a clinical parameter of the disorder; and wherein a test nucleic acid construct that provides a positive effect on a clinical parameter is a potential treatment of the disorder.

In some embodiments, the nucleotide sequence, e.g., DNA nucleotide sequence, is complementary to a portion of an mRNA that is within the translated region. In other embodiments, the nucleotide sequence, e.g., DNA nucleotide sequence, is complementary to a portion of an mRNA that is in the 5′ untranslated region (UTR) or the 3′ UTR. In some embodiments, the nucleic acid construct is an expression vector, e.g., a retroviral vector described herein, that includes the nucleotide sequence.

Evaluation methods can include methods specific for a particular disorder described herein. For example, a neurological disorder can be evaluated using electrophysiological methods (e.g., EEG and intraoperative recording), using imaging methods, and using physical, neurological and cognitive examination.

The invention also features methods of treating a subject having a neurological disorder caused by a gene, e.g., a gene described herein, by administering to a brain region of the subject a nucleic acid construct, e.g., a nucleic acid construct described herein, including a nucleotide sequence, e.g., DNA nucleotide sequence, that is complementary to a portion of an mRNA of the gene in an amount effective to inhibit translation of the mRNA, thereby treating the neurological disorder. The neurological disorders can be, e.g., obsessive compulsive disorder, schizophrenia, substance abuse, affective disorder, and Parkinson's disease.

“Cognitive function,” “cognitive process,” and “cognitive behavior” refer to the ability to think, to process and store information, and to solve problems. Typically, such processes involve specific regions of the brain, e.g., the frontal cortex, prefrontal cortex, and temporal cortex. These abilities can be distinguished from “motor function” and “motor behavior,” which involve motor areas of the brain, e.g., the motor cortex.

“Neurological disorder” refers to an impairment of cognitive function, e.g., learning and memory, due to a disease or physical trauma.

An “antisense oligonucleotide” is a nucleic acid sequence that is complementary to the sequence of a target mRNA. A “construct encoding an antisense oligonucleotide” is an expression vector containing a nucleic acid sequence that, when expressed, produces an antisense oligonucleotide.

“Identical,” as used herein in reference to nucleotide sequences, refers to the nucleotide sequence similarity between two or more nucleotide sequences. When a nucleotide position in both or all of the nucleotide sequences is occupied by the same nucleotides, then they are identical at that position. Thus, “substantially identical” means that a given nucleotide sequence is at least 90% homologous or identical with a reference sequence. In some embodiments, the given sequence can be 95, 97, 99, or even 100% identical to the reference sequence.

By “transformed cell” is meant a cell into which (or into an ancestor of which) has been introduced, by means of recombinant DNA techniques, a DNA molecule encoding (as used herein) an antisense oligonucleotide.

As used herein, the term “operably linked” means that selected DNA, e.g., encoding an antisense oligonucleotide, is in proximity with a promoter that can regulate expression of the selected DNA. Typically, the promoter is located upstream of the selected DNA in terms of the direction of transcription and translation.

By “reporter gene” is meant a gene whose expression can be assayed. Such genes include, without limitation, luciferase, β-galactoside, and green, yellow or red fluorescent protein (GFP, YFP, and RFP).

By “promoter” is meant a minimal sequence sufficient to direct transcription.

By “transgenic” is meant any cell that includes a DNA sequence that is inserted by human intervention into the cell, and would become part of the genome of an organism that develops from that cell.

By “clinical parameter” is meant any sign or symptom acknowledged by one of skill in the art to be associated with a disorder. For example, for Parkinson's disease, a clinical parameter can be one or more of tremor or trembling in hands, arms, legs, jaw, and/or face; rigidity or stiffness of the limbs and/or trunk; bradykinesia, or slowness of movement; postural instability or impaired balance and/or coordination; and/or cognitive functions, e.g., planning and/or working memory.

The invention provides several advantages. For example, the invention provides, in part, methods for the long-term, yet reversible treatment of subjects having a neurological disorder. The invention also provides, in part, methods for screening therapies and test compounds for treatment of subjects having a neurological disorder. The new methods provide simple assays for testing a variety of drug candidates that are otherwise difficult to test in vivo. The invention also provides, in part, methods for making animal models of neurological disorders. The new animal models can be designed to provide their own control, which makes testing effective and simple. Another advantage of the invention is the use of a treatment for a neurological disorder having high specificity for a particular target of the disorder.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein, including sequences reference by GenBank or other accession numbers, are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1A is a representative individual trial used in initial training of a test animal.

FIG. 1B is a representative 3-trial schedule with brightness cue. The full task consists of randomly interleaved schedules of 1, 2, and 3 trials of red-to-green color discrimination.

FIG. 1C is a representation of the five visual cue sets used in this study. Schedule states for the cues are shown at the top row.

FIG. 2 is a graph of error rates for different schedule states. Data shown here were obtained from the 2^{nd} week of testing after the reward schedules with brightness cues were introduced. Each bar represents the mean error rate averaged over all monkeys (n=7) for that schedule state. Symbols represent the error rates of individual monkeys. In 3/3 schedule state, 5 of the 7 points were very closely packed so they are difficult to distinguish on this graph.

FIG. 3A is a graph of error rates of monkeys performing the visually cued reward schedules using brightness cues in the 4^{th} week of testing after the cue's introduction before treatment. Each bar represents the mean error rate for that schedule state; the error bars are SEMs. “*” marks the conditions in which the error rates were distinguishable (single factor ANOVA, p<0.05) across the schedule states.

FIG. 3B is a graph of error rates of different groups of monkeys using length cues in the 4^{th} week after delivery of DNA constructs into the rhinal cortex. Groups of monkeys were injected with one of the following: (1) a mixture of D2 and NMDA receptor constructs (Length Cues, D2+NMDA); (2) D2 receptor construct (Length Cues, D2), (3) NMDA receptor construct (Length Cues, NMDA), or (4) vector (Length Cues, Vector Control).

FIG. 3C is a graph of error rates of the monkeys that received the D2 receptor construct after behavioral recovery. Data were obtained (approximately 12^{th} to 20^{th} weeks after injection) during the 15^{th} week after performance had recovered from the effect of either the D2 and NMDA construct mixture or D2 receptor construct alone (Length Cues, Recovered), and during the 3^{rd} week after new cues have been introduced to the same monkeys (Pattern Cues).

FIG. 3D is a graph of error rates of the monkeys that received the second treatment of D2 receptor construct (D2, n=3; and mixture of D2 and NMDA n=1). Data (Pattern Cues 2, D2) were obtained during the 8^{th} week after treatment. Data (Pattern Cues 2, Recovered) were collected during the 12^{th} week after injection, which is the 15^{th} week after performance had recovered from the effect of either the D2+NMDA receptor construct mixture or D2 receptor construct alone, and during the 3^{rd} week after new cues have been introduced to the same monkeys (Pattern Cues 3).

FIG. 4A is a representative visual cue reassignment.

FIG. 4B is an image of D2 DNA treated and normal control areas.

FIG. 4C is a representative single neuron recording from normal control area following visual cue reassignment.

FIG. 4D is a representative single neuron recording from D2 DNA treated area following visual cue reassignment.

FIG. 5A is an autoradiograph of a single brain section from the monkey treated with DNA construct targeting D2 receptor protein showing D2 receptor binding using [¹²⁵I]-Iodosulpride. The rhinal cortex in the left hemisphere (between the two arrows) was treated by DNA targeting the D2 receptor. “rs” indicates rhinal sulcus, “amts” indicates anterior middle temporal sulcus, and “A” marks the Amygdala.

FIG. 5B is a graph of the means and standard errors of the mean for the average density of D2 receptors in the D2 receptor construct treated rhinal cortex (treated) and in the untreated rhinal cortex (control). “*” indicates a significant difference between the two hemispheres (paired t-test, p=0.001).

FIG. 5C is a graph of NMDA receptor density in the rhinal cortex of NMDA targeting DNA construct treated monkey. “*” indicates a significant difference between the two hemispheres (paired t-test, p=0.003).

FIG. 5D is a graph of D2 receptor density in the rhinal cortex of NMDA targeting DNA construct treated monkey. NMDA treatment did not affect the D2 receptor (paired t-test, p=0.6).

### DETAILED DESCRIPTION

Certain neurological disorders involve the expression of specific proteins. The invention is based, at least in part, on the discovery that nucleic acid constructs that include nucleotide sequences, e.g., oligodeoxynucleotide antisense sequences, can be administered to a specific brain region to affect cognitive behavior. In particular, the administration of a nucleic acid construct encoding an antisense sequence that targets the D2 dopamine receptor, to the rhinal cortex of a primate brain was found to impair the ability of primates to use visual cues to predict the amount of work to be completed to obtain a reward. Administration of the D2 antisense nucleic acid construct was also found to affect the protein levels of the D2 dopamine receptor in the rhinal cortex. Further, the nucleic acid construct-mediated impairment was found to be long-lasting (10 to 15 weeks), yet reversible. Accordingly, the invention encompasses methods and therapeutic compositions for treatment of neurological disorders, and methods of screening test compounds and potential therapies for treatment of various neurological disorders, as well as methods of producing animal models of neurological disorders, and the models themselves.

Selecting Gene Targets for Antisense

The methods and compositions described herein can be used to target proteins implicated in various neurological disorders. The targets and their associated disorders can include, e.g., the D2 dopamine receptor (e.g., GenBank Accession Nos. M90314.1; AF358821.1; NM_{—}012547.1; NM_{—}010077.1; AB080609.1), for obsessive compulsive disorder, schizophrenia, and drug abuse; DEP1 and dopamine P-hydroxylase (e.g., GenBank Accession Nos. S50200.1; BC017174.2; AF070919.1; L12407.1; NM_{—}013158.1) for affective disorder; neuregulin 1 (e.g., GenBank Accession Nos. CR857875.1; NM_{—}031588.1; NM_{—}013964.1; BC073871.1; XM_{—}486093.1), dystrobrevin binding protein (e.g., GenBank Accession Nos. NM_{—}032122.3; NM_{—}183041.1; NM_{—}183040.1; NM_{—}025772.3; BC058574.1) and disrupted in schizophrenia-1 (DISC-1) (e.g., GenBank Accession Nos. NM 175596.2; NM_{—}174854.1; NM_{—}174853.1; NM_{—}170596.1; NM_{—}018662.1; AY320287.1; AY177674.2; AY177673.1) for schizophrenia; and the 5-HT1A receptor (e.g., GenBank Accession Nos. NM_{—}000524.2; NM_{—}012585.1; BC069159.1; AK049884.1; AK049814.1; U39391.1; NM_{—}008308.2), the δ-opioid receptor (e.g., GenBank Accession Nos. NM_{—}012617.1; NM_{—}013622.2; NM_{—}000911.2; AK043873.1; U07882.2; L07271.1; L11064.1; U00475.1), the GABA(A) receptor (e.g., GanBank Accession Nos. NM_{—}000808.2; NM_{—}183326.1; NM_{—}008070.2; NM_{—}010250.2; NM_{—}008073.1; NM_{—}177408.2; NM_{—}000814.3; NM_{—}021912.2; NM_{—}000812.2; M62400.1), the β2-adrenergic receptor (e.g., GenBank Accession Nos. BC086538.1; NM_{—}012492.2; NM_{—}007420.2; NM_{—}000024.3; NM_{—}174231.1; BC032883.1; AY136741.1; L38905.1; Z86037.1; M15169.1), and/or the GluR2 receptor (e.g., GenBank Accession No. AF164344.1; L35318.1; M85035.1; X64830.1) for alcohol addiction (see, e.g., Colangelo et al., J. of Neurosci. Res., 70:462-473 (2002); Newton et al., J. of Neurosci., 23:10841-10851 (2003); Li et al., Molecular Neurobiol., 25:265-285 (2002); Hoffman et al., Alcohol Clin. Exp. Res., 27:155-168 (2003)).

Antisense Oligonucleotides

The methods and compositions described herein employ antisense compounds, particularly antisense oligonucleotides, for use in modulating the function of nucleic acid molecules encoding a protein, e.g., a D2 dopamine receptor, ultimately modulating the amount of the protein, e.g., a D2 dopamine receptor, produced. This can be accomplished by providing antisense oligonucleotides that specifically hybridize with nucleic acids, preferably mRNA, encoding a protein, e.g., a D2 dopamine receptor.

The methods and compositions described herein involve the design of antisense oligonucleotides that are complementary to an mRNA encoding a protein of interest. General approaches to constructing oligonucleotides useful in antisense therapy have been reviewed, for example, by Van der Krol et al., Biotechniques, 6:958-976 (1988); and Stein et al., Cancer Res., 48:2659-2668 (1988). The antisense oligonucleotides can be oligodeoxynucleotides and can inhibit expression of a protein, for example, by binding to mRNA transcripts and preventing translation. Absolute complementarity, although preferred, is not required. The ability to hybridize will depend on both the degree of complementarity and the length of the antisense nucleic acid. Generally, the longer the hybridizing nucleic acid, the more base mismatches with an RNA it can contain and still form a stable duplex (or triplex, as described herein). The degree of mismatch allowed can be ascertained by using standard procedures to determine the melting point of the hybridized complex.

Endogenous gene expression can also be reduced by targeting deoxyribonucleotide sequences complementary to the regulatory region, i.e., the promoter and/or enhancers, of a gene to form triple helical structures that prevent transcription of the target gene. (See generally, Helene, Anticancer Drug Des., 6:569-584 (1991); Helene et al., Ann. N.Y. Acad. Sci., 660:27-36 (1992); and Maher, Bioassays, 14:807-815 (1992)).

Selecting the mRNA Hybridization Region

In accordance with the methods and compositions described herein, “mRNA” denotes not only informational ribonucleotide sequences that encode a protein using the three letter genetic code, but also associated ribonucleotide sequences that form a region such as the 5′-untranslated region, the 3′-untranslated region, the 5′ cap region and intron/exon junction ribonucleotide sequences. Thus, antisense oligonucleotides can be formulated that are targeted wholly or in part to these associated ribonucleotide sequences as well as to the informational ribonucleotide sequences. The antisense oligonucleotide can therefore specifically hybridize to a transcription initiation site region, a translation initiation codon region, a 5′ cap region, an intron/exon junction, coding sequences, a translation termination codon region, or sequences in the 5′- or 3′-untranslated region. Since the translation initiation codon is typically 5′-AUG (in transcribed mRNA molecules; 5′-ATG in the corresponding DNA molecule), the translation initiation codon is also referred to as the “AUG codon,” the “start codon,” or the “AUG start codon.” A minority of genes have a translation initiation codon having the RNA sequence 5′-GUG, 5′-UUG, or 5′-CUG. Additionally, 5′-AUA, 5′-ACG, and 5′-CUG have been shown to function in vivo.

Thus, the terms “translation initiation codon” and “start codon” can encompass many codon sequences, even though the initiator amino acid in each instance is typically methionine (in eukaryotes) or formylmethionine (prokaryotes). It is also known in the art that eukaryotic and prokaryotic genes can have two or more alternative start codons, any one of which can be preferentially utilized for translation initiation in a particular cell type or tissue, or under a particular set of conditions.

In the context of the methods and compositions described herein, “start codon” and “translation initiation codon” refer to the codon or codons that are used in vivo to initiate translation of an mRNA molecule transcribed from a gene encoding a protein, e.g., D2 dopamine receptor, regardless of the sequence(s) of such codons. It is also known that a translation termination codon (or “stop codon”) of a gene can have one of three sequences, i.e., 5′-UAA, 5′-UAG and 5′-UGA (the corresponding DNA sequences are 5′-TAA, 5′-TAG, and 5′-TGA, respectively). The terms “start codon region,” “AUG region,” and “translation initiation codon region” refer to a portion of such an mRNA or gene that encompasses from about 25 to about 50 contiguous nucleotides in either direction (i.e., 5′ or 3′) from a translation initiation codon. This region is a useful target region for antisense oligonucleotides. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. This region is also a useful target region for antisense oligonucleotides.

The open reading frame (ORF) or “coding region,” which is known to refer to the region between the translation initiation codon and the translation termination codon, is also a region that can be targeted effectively. Antisense oligonucleotides can target the entire coding region, or a portion thereof.

Other useful target regions include the 5′ untranslated region (5′UTR), which refers to the portion of an mRNA in the 5′ direction from the translation initiation codon, and thus includes nucleotides between the 5′ cap site and the translation initiation codon of an mRNA, or corresponding nucleotides on the gene, and the 3′ untranslated region (3′UTR, Wagner, Nature, 372:333-335 (1994)), which refers to the portion of an mRNA in the 3′ direction from the translation termination codon, and thus includes nucleotides between the translation termination codon and 3′ end of an mRNA, or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA includes the 5′ cap structure itself as well as the first 50 nucleotides adjacent to the cap. The 5′ cap region can also be a suitable target region for antisense oligonucleotides.

Although some eukaryotic mRNA transcripts are directly translated, many contain one or more regions, known as “introns,” which are excised from the pre-mRNA transcript to yield one or more mature mRNAs. The remaining (and therefore translated) regions are known as “exons” and are spliced together to form a continuous mRNA sequence. mRNA splice sites, i.e., exon-exon or intron-exon junctions, can also be useful target regions, and are particularly useful in situations where aberrant splicing is implicated in disease, or where an overproduction of a particular mRNA splice product is implicated in disease. Aberrant fusion junctions due to rearrangements or deletions are also suitable targets. Targeting particular exons in alternatively spliced mRNAs can also be useful. It has also been found that introns can also be effective target regions for antisense oligonucleotides targeted, for example, to DNA or pre-mRNA.

Alternatively, one of skill in the art can choose and synthesize any of a number of appropriate antisense oligonucleotides for use in accordance with the present methods using, e.g., “gene walk.” A “gene walk” comprising a series of oligonucleotides of 15-30 nucleotides spanning the length of a target gene can be prepared, followed by testing for inhibition of gene expression. Optionally, gaps of 5-10 nucleotides can be left between the oligonucleotides to reduce the number of oligonucleotides synthesized and tested.

Determining the Antisense Oligonucleotide Sequence

Once the target site or sites have been identified, antisense oligonucleotides are designed that are sufficiently complementary to the target, i.e., hybridize sufficiently well and with sufficient specificity, to give the desired modulation.

An antisense oligonucleotide sequence specifically hybridizes when binding of the sequence to the target DNA or RNA molecule interferes with the normal function of the target DNA or RNA to cause a loss of activity, and there is a sufficient degree of complementarity to avoid non-specific binding of the antisense sequence to non-target sequences under conditions in which specific binding is desired, i.e., under physiological conditions in the case of in vivo assays or therapeutic treatment, and in the case of in vitro assays, under conditions in which the assays are performed. The antisense sequences are at least 80% complementary to the target mRNA, e.g., at least 90%, 98%, 99%, or 100% complementary. Percent complementarity of an antisense compound with a target mRNA can be determined using routine tools, such as the Basic Local Alignment Search Tool (BLAST) (Altschul et al., J. Mol. Biol., 215:403-410 (1990); Zhang et al., Genome Res., 7:649-656 (1997)).

In one embodiment, the antisense sequence is complementary to relatively accessible sequences of the mRNA (e.g., relatively devoid of secondary structure). These sequences can be determined by analyzing predicted RNA secondary structures using, for example, the MFOLD program (Genetics Computer Group, Madison Wis.) and testing in vitro or in vivo as is known in the art. Other useful methods for identifying effective antisense compositions can include combinatorial arrays of oligonucleotides (see, e.g., Milner et al., Nature Biotechnology, 15:537-541 (1997)).

Methods of Making Antisense Oligonucleotides

Antisense oligonucleotides described herein can be prepared by routine methods for the synthesis of DNA and RNA molecules. These include techniques for chemically synthesizing oligodeoxyribonucleotides (e.g., by use of an automated DNA synthesizer such as is commercially available from Biosearch, Applied Biosystems, etc.) and oligoribonucleotides (e.g., by solid phase phosphoramide chemical synthesis).

An antisense oligonucleotide can be chemically synthesized as described, for example, in Beaucage et al., Tetra. Letts., 22:1859-1862 (1981); and Matteucci et al., J. Am. Chem. Soc., 103:3185-3191 (1981). Antisense oligonucleotides can by synthesized using naturally occurring nucleotides or variously modified nucleotides designed to increase the biological stability of the molecules or to increase the physical stability of the duplex formed between the antisense and sense nucleic acids. For example, phosphorothioate, phosphoramidate, and methylphosphonate derivatives of nucleotides can be used (see, e.g., U.S. Pat. Nos. 5,176,996; 5,264,564; and 5,256,775). Acridine substituted nucleotides can also be used. The most widely used modified antisense oligonucleotides are phosphorothioates, where one of the oxygen atoms in the phosphodiester bond between nucleotides is replaced with a sulfinur atom. These phosphorothioate antisense oligonucleotides have greater stability in biological fluids than normal oligos and are preferred antisense nucleic acids within the invention. As examples, phosphorothioate oligonucleotides can be synthesized, e.g., by the method of Stein et al. (Nucl. Acids Res., 16:3209-3221 (1988)), and methylphosphonate oligonucleotides can be prepared, e.g., by use of controlled pore glass polymer supports (Sarin et al., Proc. Natl. Acad. Sci. U.S.A., 85:7448-7451 (1988)).

The antisense oligonucleotides also can be produced biologically by application of recombinant DNA techniques. Standard reference works setting forth the general principles of recombinant DNA technology and cell biology, which are hereby incorporated by reference, include Watson et al., Molecular Biology of the Gene, Volumes I and II, Benjamin/Cummings Publishing Co., Inc., Menlo Park, Calif. (1987); Darnell et al., Molecular Cell Biology, Scientific American Books, Inc., New York, N.Y (1986); Lewin, Genes II, John Wiley & Sons, New York, N.Y (1985); Old et al., Principles of Gene Manipulation: An Introduction to Genetic Engineering, 2nd Ed., University of California Press, Berkeley, Calif. (1981); Maniatis et al., Molecular Cloning: A Laboratory Manual, Cold Spring Harbor Press, Cold Spring Harbor, N.Y. (1982); Sambrook et al., Molecular Cloning: A Laboratory Manual, 2nd Edition, Cold Spring Harbor Press, Cold Spring Harbor, N.Y. (1989); and Albers et al., Molecular Biology of the Cell, 2nd Ed., Garland Publishing, Inc., New York, N.Y (1989). Techniques for synthesizing such molecules are disclosed by, for example, Wu et al., Prog. Nucl. Acid. Res. Molec. Biol., 21:101-141 (1978). Procedures for constructing recombinant molecules are disclosed in detail by Sambrook et al. (supra).

Typically, a cDNA of a gene of interest is cloned from a library, e.g., a genomic library. The cDNA, or a portion of the cDNA, is then cloned into an expression vector. An “expression vector” is a vector that (due to the presence of appropriate transcriptional and/or translational control sequences) is capable of expressing a DNA (or cDNA) molecule that has been cloned into the vector and of thereby producing a polypeptide or protein. Expression of the cloned sequences occurs when the expression vector is introduced into an appropriate host cell, e.g., in a particular tissue or organ, such as the brain, in the host (e.g., a mammalian, e.g., human, subject). An appropriate mammalian host cell would be any mammalian cell capable of expressing the cloned sequences. Procedures for preparing cDNA and for producing a genomic library are disclosed by Sambrook et al. (supra).

The cDNA, or a portion of the cDNA, can be cloned into an expression vector in accordance with routine techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. For use in the methods described herein, the resulting expression vector, or nucleic acid construct, contains a nucleic acid inserted in an antisense orientation (i.e., RNA transcribed from the inserted nucleic acid will be of an antisense orientation relative to the target nucleic acid of interest, e.g., mRNA). Techniques for such manipulations are disclosed by Sambrook et al. (supra).

The antisense expression vector, or nucleic acid construct, can be in the form of a recombinant plasmid, phagemid, or attenuated virus. Routine methods can be used to obtain suitable antisense vectors (see, e.g., Mautino et al., Hum. Gene Ther., 13:1027-1037 (2002); Mautino et al., Gene Ther., 9:421-431 (2002); Mautino et al., AIDS Patient Care STDS, 16:11-26 (2002); Pachori et al., Hypertension, 39:969-975 (2002)). Conventional methods of gene transfer and gene therapy can also be adapted for use in the present invention. See, e.g., Gene Therapy: Principles and Applications, ed. Blackenstein, Springer Verlag, 1999; Gene Therapy Protocols (Methods in Molecular Medicine), ed. Robbins, Humana Press, 1997; and Retro-vectors for Human Gene Therapy, ed. Hodgson, Springer Verlag, 1996.

Nucleic acid constructs can include a nucleotide sequences other than those encoding an antisense oligonucleotide. For example, the nucleic acid construct can include a nucleotide sequence encoding a reporter protein, e.g., green, yellow, or red fluorescent protein (GFP, YFP, or RFP). Both nucleotide sequences can be operably linked to a single promoter, or they can be operably linked to separate promoters, such that expression of the reporter protein indicates or signals expression of the antisense oligonucleotide.

Determining the Efficacy of an Antisense Oligonucleotide

In vitro studies can be performed to quantify the ability of the antisense oligonucleotide to inhibit gene expression. These studies can utilize controls that distinguish between antisense gene inhibition and nonspecific biological effects of oligonucleotides. These studies can also compare levels of the target RNA or protein with that of an internal control RNA or protein. Additionally, results obtained using the antisense oligonucleotide can be compared with those obtained using a control oligonucleotide. Preferably, the control oligonucleotide is of approximately the same length as the antisense oligonucleotide and the nucleotide sequence of the control oligonucleotide differs from the antisense oligonucleotide no more than is necessary to prevent specific hybridization of the control to the target sequence.

Expression patterns within cells or tissues treated with one or more antisense oligonucleotides can be compared to control cells or tissues not treated with antisense oligonucleotides and the patterns produced can be analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds that affect expression patterns.

Examples of routine methods of gene expression analysis include DNA arrays or microarrays (Brazma et al., FEBS Lett., 480:17-24 (2000); Celis et al., FEBS Lett., 480:2-16 (2000)), SAGE (serial analysis of gene expression) (Madden et al., Drug Discov. Today, 5:415-425 (2000)), READS (restriction enzyme amplification of digested cDNAs) (Prashar et al., Methods Enzymol., 303:258-272 (1999)), TOGA (total gene expression analysis) (Sutcliffe et al., Proc. Natl. Acad. Sci. U.S.A., 97:1976-1981 (2000)), protein arrays and proteomics (Celis et al., FEBS Lett., 480:2-16 (2000); Jungblut et al., Electrophoresis, 20:2100-2110 (1999)), expressed sequence tag (EST) sequencing (Celis et al., FEBS Lett., 480:2-16 (2000); Larsson et al., J. Biotechnol., 80:143-157 (2000)), subtractive RNA fingerprinting (SuRF) (Fuchs et al., Anal. Biochem., 286:91-98 (2000); Larson et al., Cytometry, 41:203-208 (2000)), subtractive cloning, differential display (DD) (Jurecic et al., Curr. Opin. Microbiol., 3:316-321 (2000)), comparative genomic hybridization (Carulli et al., J. Cell Biochem. Suppl., 31:286-296 (1998)), FISH (fluorescent in situ hybridization) techniques (Going et al., Eur. J. Cancer, 35:1895-1904 (1999)) and mass spectrometry methods (reviewed in To, Comb. Chem. High Throughput Screen, 3:235-241 (2000)).

Such expression analysis can be used to determine an optimal concentration of an antisense oligonucleotide, or of a nucleic acid construct encoding an antisense oligonucleotide, to inhibit gene expression. An optimal concentration can be determined by administering different concentrations of the antisense oligonucleotide and monitoring the effect on gene expression.

Methods of Administering Antisense Oligonucleotides

To prepare a nucleic acid construct encoding an antisense sequence for administration, the nucleic acid construct can be suspended in a medium to facilitate transfection into cells using routine techniques. For example, the nucleic acid construct can be suspended in artificial cerebrospinal fluid and combined with a transfection material, e.g., a lipid, e.g., DOTAP (1,2-dioleoyl-3-trimethy-ammonium propane; Avanti Polar Lipids).

In some embodiments, a nucleic acid construct encoding an antisense oligonucleotide is administered to a subject, e.g., is administered to a particular region of the body, e.g., a brain region. For injection into the brain, known imaging and stereotaxic equipment can be used. The imaging can be performed using direct visualization with a surgical microscope, or can be performed using a camera and/or a computer. The method of introduction of the antisense oligonucleotide into the brain can include any routine physical method of introducing material into the brain parenchyma, an anatomical region of the CNS or the cerebrospinal fluid. Such methods include, e.g., viral delivery, targeted delivery using liposomes, and direct injection using a miniosmotic pump, a needle, a syringe, or similar mechanism. Preferably, the antisense oligonucleotide is injected using a needle, e.g., a 30-gauge needle, and a syringe, e.g., a 10 μL syringe.

Therapeutic Uses of Antisense Oligonucleotides

A nucleic acid construct encoding an antisense oligonucleotide described herein can be administered as a therapeutic agent to a subject, e.g., a mammal, e.g., a human, exhibiting a neurological disorder. The methods can be used to treat disorders including impairments or clinical parameters that are associated with or caused by excessive levels of a target protein or mRNA. The targets of antisense oligonucleotides and their associated disorders can include, e.g., the D2 dopamine receptor for obsessive compulsive disorder, schizophrenia, and drug abuse; DEP1 and dopamine β-hydroxylase for affective disorder; neuregulin 1, dystrobrevin binding protein and disrupted in schizophrenia-1 (DISC-1) for schizophrenia; and the 5-HT1A receptor, the 6-opioid receptor, the GABA(A) receptor, the P2-adrenergic receptor, and the GluR2 receptor for alcohol addiction (see, e.g., Colangelo et al., J. Neurosci. Res., 70:462-473 (2002); Newton et al., J. Neurosci., 23:10841-10851 (2003); Li et al., Molecular Neurobiol., 25:265-285 (2002); Hoffman et al., Alcohol Clin. Exp. Res., 27:155-168 (2003)).

The nucleic acid construct can be administered, as described herein, to a specific region of the body, e.g., an organ, e.g., the brain. The selection of the region for administration can be made based on the target, e.g., protein, for a particular disorder, as described herein. Following the administration of the antisense oligonucleotide, the expression of the targeted protein can be analyzed as described herein. The subject, e.g., a human, can then be evaluated for changes in the disorder. Evaluation methods specific for a particular disorder can be employed. Neurological disorders can be evaluated, e.g., clinically, e.g., using electrophysiological methods (e.g., EEG and intraoperative recording), using imaging methods, and using physical, neurological, and/or cognitive examination.

The methods described herein can be used to temporarily modify the expression of a target protein. Following the initial administration, the subject can be evaluated periodically, e.g., daily, for effects of the administration of the nucleic acid construct on the disorder. Following attenuation of the modification induced by the nucleic acid construct, a different concentration of the nucleic acid construct can be administered and the subject can be reevaluated. Alternatively, a nucleic acid construct encoding an antisense oligonucleotide directed to a different protein target can be administered to the subject, followed by reevaluation.

For administration into humans, the nucleic acid construct can be used at doses similar to those used in the examples described herein. For example, the nucleic acid construct can be used at a concentration of at least 1 μg, e.g., at least 5, 10, 20, 30, 40, 50, 100, 200 and 500 μg. The nucleic acid construct can be administered, e.g., at a volume of at least 0.1 μL, e.g., at least 0.2, 0.3, 0.5, 0.75, 1, 1.5, 2, 4, 10, and 25 μL, per injection. The concentration of nucleic acid construct to be administered can be determined by evaluating the effects of different concentrations of the nucleic acid construct on protein expression, as described herein.

Screening Methods Using Antisense Constructs

The methods and compositions described herein can be used to screen test compounds for their ability to treat a specific disorder. In particular, the methods and compositions can be used to test the efficacy and specificity of a treatment for a particular neurological disorder.

A subject, e.g., a mammal, e.g., a human, non-human primate, horse, cat, or dog, having a particular disorder, e.g., a neurological disorder, can be first treated with a test compound. The effect of the test compound on the disorder can then be evaluated as described herein and the treatment can be altered accordingly to treat the disorder. An antisense oligonucleotide described herein can then be administered to a particular body region, e.g., the brain, in an amount sufficient to effectively modify, e.g., reduce, the expression of a target protein. The efficacy of the test compound for treating the disorder can then be evaluated. This method can be used to determine whether a test compound, previously determined to effectively treat a disorder, is effective following the modulation of the expression of a particular protein, thereby evaluating the efficacy and specificity of a test compound for a particular disorder.

The methods and compositions described herein can also be used to screen potential therapies for a disorder. A nucleic acid construct encoding an antisense oligonucleotide described herein can be administered to a healthy subject to induce a specific disorder in the subject. A test therapy can then be administered to the subject. Such therapies can include, e.g., the administration of pharmaceuticals (e.g., L-DOPA and antibiotics), surgical treatment, radiotherapy, and psychotherapy. The effect of the therapy on the induced disorder can be evaluated, and the therapy can be altered to effectively treat the induced disorder.

The methods and compositions described herein can also be used to screen potential treatments for a particular disorder. For example, the methods and compositions described can be used to temporarily modulate protein expression in a particular region of the body and evaluate the effect on the disorder. Following the determination of a particular protein and/or a particular body region, a more permanent treatment focused on that particular protein and/or body region can be used. Such permanent treatments can include, e.g., surgical removal of tissue from the body region, the use of viral vectors, and the use of noncompetitive inhibitors.

Methods of Making Animal Models of Neurological Disorders

The methods and compositions described can also be used to make animal models of human disorders, e.g., schizophrenia, use/abuse of cocaine, and Parkinson's disease. These animal models can be used to test compounds and therapies for treatment of the human disorder, e.g., Parkinson's disease.

Parkinson's disease is primarily a motor system motor system disorder resulting in the loss of dopamine producing brain cells. Loss of dopamine leaves patients unable to direct or control their movement in a normal manner. The four primary symptoms of Parkinson's are tremor or trembling in hands, arms, legs, jaw, and face; rigidity or stiffness of the limbs and trunk; bradykinesia, or slowness of movement; and postural instability or impaired balance and coordination. In addition to motor impairments, Parkinson's disease impairs cognitive functions, e.g., planning and working memory (see, e.g., Lewis et al., J. Neurosci., 23:6351-6356 (2003); Higginson et al., Brain and Cognition, 52:343-352 (2003)). These cognitive deficits resemble those produced by damage to the frontal cortex of the brain.

Parkinson's disease can be induced in an animal, e.g., a monkey, by administering a nucleic acid construct encoding an antisense oligonucleotide described herein to a particular brain region. The nucleic acid construct can target a gene encoding any of the proteins involved in the dopamine pathway, e.g., a dopamine receptor and the enzyme tyrosine hydroxylase. The nucleic acid construct can be administered to the brain bilaterally or unilaterally. A unilaterally treated monkey, with Parkinson's disease induced on only one side of the brain, can be used to test the efficacy of a compound in treating the disorder by comparing the effects of the compound on the activity of the Parkinson's disease-induced side of the brain to the effects on the activity of the normal side of the brain. Thus, the monkey serves as its own control.

This so-called “Hemi-Parkinsonism” monkey model can be used to test compounds that are potential candidates to treat motor and/or cognitive deficits associated with Parkinson's disease. The effects of the test compounds can be evaluated clinically, e.g., by monitoring electrical activity of the brain or by monitoring the symptoms, e.g., the motor and cognitive deficits.

### EXAMPLES

The invention is further described in the following examples, which do not limit the scope of the invention described in the claims.

### Example 1

### Effect of Unilateral Rhinal Cortex Ablation on Behavior

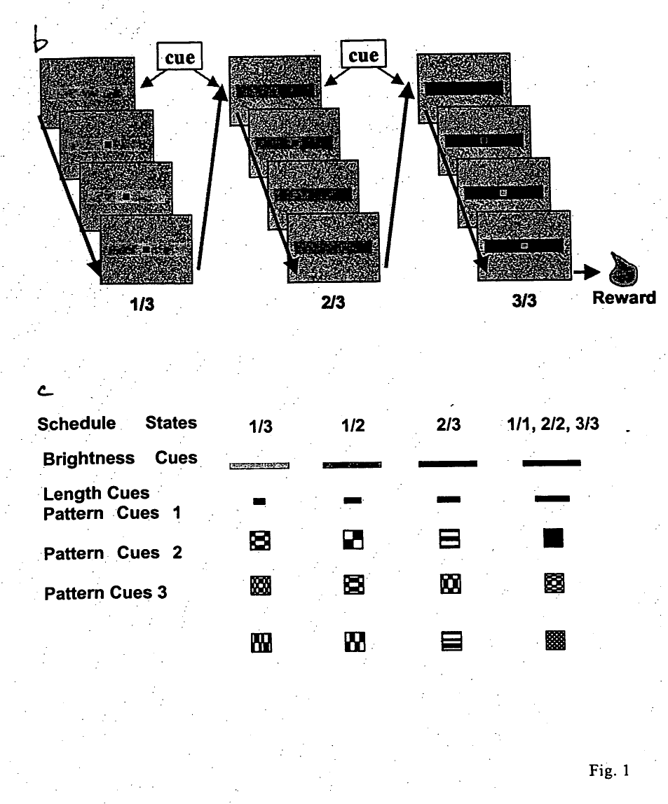

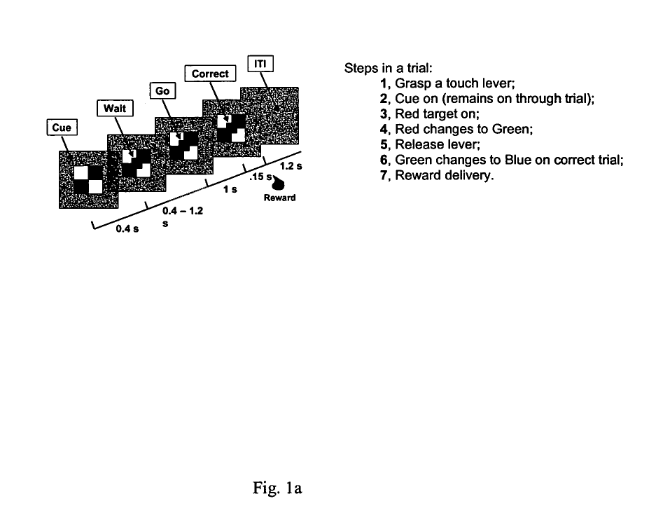

In the behavioral task used here, visually cued reward schedules (Bowman et al., J Neurophysiol., 75:1061-1073 (1996); Liu et al., Nature Neurosci., 3:1307-1315 (2000); Liu et al., J. Neurophysiol., 83:1677-1692 (2000); Shidara et al., Science, 296:1623-1624 (2002)), the monkeys are exposed to several visual cues, each of which is related to the workload yet to be completed before reward delivery. The workload is defined as the number of color discrimination trials that must be performed. In the task as used here, a schedule of 1, 2, or 3 color discrimination trials must be performed successfully to obtain a liquid reward (FIG. 1; Bowmen et al., supra; Liu et al., supra). Progress to the next trial in the schedule only occurs after a correct trial with no explicit punishment given for an incorrect response. After reward delivery, the monkey is presented with a new randomly chosen schedule with 1, 2, or 3 trials. When normal monkeys perform this task, the number of errors is related to the workload remaining before a reward. The monkeys make progressively fewer errors on trials closer to the reward, with the fewest errors in the rewarded trial (Bowman et al., supra; Liu et al., supra; Liu et al., supra; Shidara et al., supra). For convenience in identifying trial types in the task, we labeled each trial type by its position in a schedule (schedule state), which is the current trial number/current schedule length (e.g., 1/3, 2/3, and 3/3 for the 1st, 2nd, and 3rd trial in a 3-trial schedule).

Rhinal Cortex Ablations

Each rhesus monkey (n=7) was first given a unilateral rhinal cortex ablation before any behavioral training, thereby limiting the need for DNA injection into the rhinal cortex to one hemisphere (Table 1). For the unilateral rhinal cortex ablations, surgery was performed as previously described (Meunier et al., J. Neurosci., 13:5418-5432 (1993)). For all 7 monkeys, the location and extent of the cortical removals were evaluated using magnetic resonance images (MRI) (Liu et al., supra; Bachevalier et al., Behav. Neurosci., 113:1127-1151 (1999)). The unilateral lesions of the rhinal cortex were essentially as intended, being of similar size and location to those reported in our other study (Liu et al., supra). Quantitative assessments were done on 6 monkeys. The lesions removed 85.2%±3.0% (mean±SEM, n=6) of the rhinal cortex. The quality of the MRI was inadequate to assess the size of the lesion in the seventh monkey.

Visually Cued Reward Schedules

Two weeks after the unilateral rhinal cortex removal, the monkeys were trained on a red-to-green color discrimination task (FIG. 1a). The full task consisted of randomly interleaved schedules of 1, 2, and 3 trials of red-to-green color discrimination. On each trial, a visual cue indicated progress through the schedule. Monkeys were seated in a primate chair facing a rear projection screen (90°×90°) located 57 cm away. A touch lever was available to register the monkey's responses. A black and white random dot background covered the whole screen. Both a visual cue and a colored spot were shown at the center of the screen. In each of the discrimination trials the monkey was required to release a touch lever when a spot (0.5°) on the screen changed color from red to green.

In a 3-trial schedule (FIG. 1b), the monkey was required to correctly perform 3 of the color discrimination trials to obtain a reward at the end of the third trial. Each trial was assigned a schedule state showing the amount of work remaining before the rewarded trial (current trial number/schedule length, e.g., 1/3 for the 1st trial, 2/3 for the 2^{nd} trial, and 3/3 for the 3^{rd} trial of a 3-trial schedule). The monkey started each trial by grasping a touch lever. Immediately after the lever was contacted, a visual cue (e.g., the rectangle in the middle of each screen in FIG. 1b) was displayed and remained on throughout the whole trial. In FIG. 1b, the light gray rectangle was the cue for schedule state 1/3, the dark gray rectangle was the cue for 2/3, and the black rectangle was the cue for 3/3. The cue was displayed alone for 400-500 ms, and a red spot then appeared at the center of the screen. After a randomly selected wait time (400, 600, 800, 1000, or 1200 ms), the color of the spot changed from red to green, indicating that the monkey could release the lever to complete a trial. If the monkey released the lever within 1000 ms after the spot turned to green, indicating that the monkey had detected this color change, the spot changed from green to blue and displayed for 150 ms, signaling that the trial had been performed correctly. All stimuli then disappeared. If the trial was the last in a schedule (e.g., 3/3), a liquid reward was delivered. Each trial was separated by a 1200 ms intertrial interval. If the monkey released the lever during the red spot period or in less than 200 ms after the onset of the green spot, or if the monkey did not release the lever within 1000 ms after the onset of the green spot, all stimuli disappeared, the trial was terminated, and an error was registered. There was no explicit punishment for errors; the same cue reappeared in the next trial, and the monkey still needed to complete the requisite number of correct trials for that schedule before a reward was delivered. A new schedule was chosen pseudorandomly after the completion of the previous schedule. There was no requirement for the monkey either to notice or to use the cues in the task.

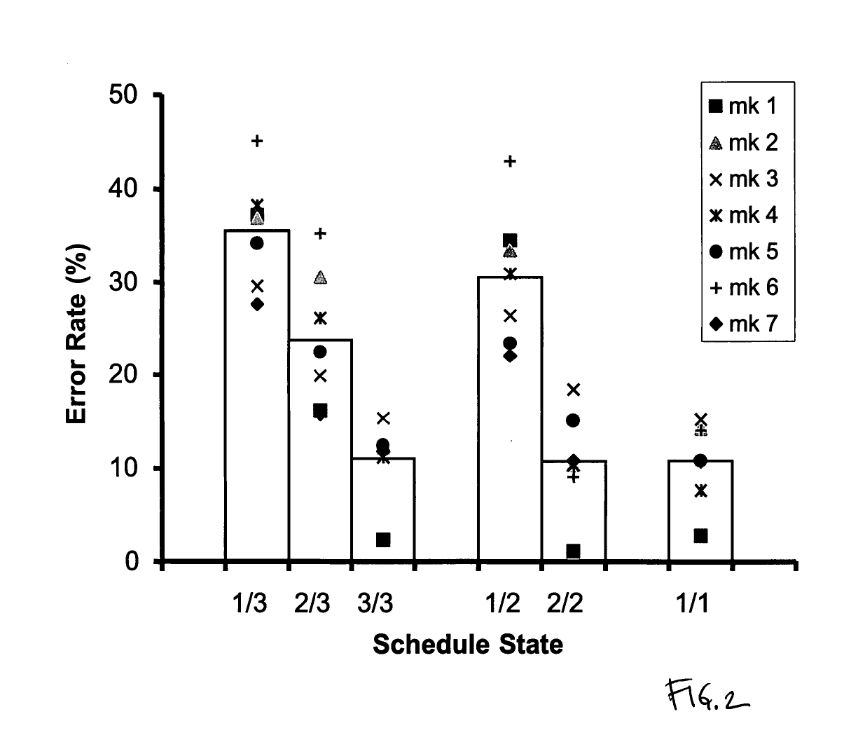

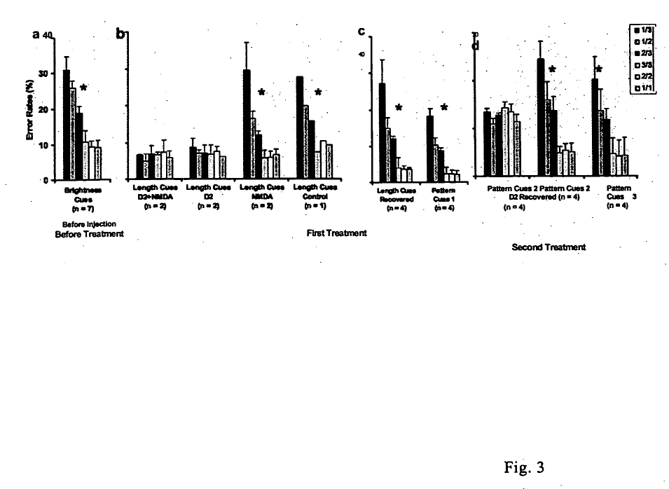

During initial training, every correctly performed trial was rewarded with a drop of liquid (see FIG. 1a). Within two weeks after starting behavioral training all seven monkeys with unilateral lesions performed ˜90% of color discrimination trials correctly in two consecutive test sessions, a rate of acquisition no different than that of intact monkeys (Liu et al., supra). At this point the visually cued reward schedules were abruptly introduced. The monkeys were initially exposed to cues that varied in brightness (brightness cues, FIG. 1c; for pre-injection testing, all monkeys were tested for 4 consecutive weeks (4 days/week). Monkeys were allowed to perform as many trials as they wanted in each session, normally between 600 and 1000 trials.) As expected, by the second week after introduction of the reward schedules, the number of errors scored by each monkey was directly related to the number of trials remaining before reward delivery (FIG. 2). The monkeys made progressively fewer errors as the workload remaining before reward became smaller (on trials closer to reward), with the fewest errors occurring in the final, rewarded trial of each schedule. For each of the 7 monkeys, the error scores were significantly different across the three nonrewarded states, i.e., 1/3, 1/2, and 2/3 states (˜2 test, p<0.05 for each monkey). Data from each week were combined for analysis. Performance of each individual monkey was evaluated using ˜2 test on the numbers of correct and incorrect trials. Group analysis was tested using repeated measures ANOVA with percent of errors (error rate) from each monkey in each group. Difference in receptor binding density was tested using paired t-test (one-tailed). All statistics were evaluated at 0.05 levels. The error scores were statistically indistinguishable in all rewarded schedule states (1/1, 2/2, 3/3 states), no matter which schedule (1, 2, or 3-trial) was in effect (˜2 test, p>0.05). The error score for each of the schedule states was significantly different among the trials in a schedule (˜2 test, p<0.05). For the group, the relation between the averaged error rates and schedule states remained the same from the second to the fourth week of testing (interaction term of a two way ANOVA, F_{10,125}=0.329, p=0.97; FIG. 2; FIG. 3a).

Because visual cues provide the only source of information about the number of trials to be completed before reward delivery, this pattern of errors indicates that the monkeys used the visual cues to predict the workloads. The patterns of learning and performance of all 7 monkeys with unilateral rhinal cortex removals trained on the reward schedule task were similar to those observed in intact monkeys (Liu et al., supra; Akil et al., Cerebral Cortex, 3:533-550 (1993); Shidara et al., supra; Bowman et al., supra; Gaffan et al., Behav. Brain Res., 3:149-163 (1988); Parker et al., Neuropsychologia, 36:259-271 (1998); Ettlinger et al., J. Comp. Physiol., 65:110-117 (1968), e.g., the learning and performance were indistinguishable from the initial learning scores of the 5 monkeys in prior ablation studies (interaction term of a two way ANOVA, F_{5,71}=1.74, p=0.14; Liu et al., supra). Because the monkeys learned the reward schedules at a normal rate after the unilateral surgical lesion, these results indicate that one intact rhinal cortex is sufficient to support this learning.

### Example 2

### Effect of Antisense Oligonucleotide Administration to Rhinal Cortex on Behavior

Here, we injected adult monkey rhinal cortex with DNA antisense expression constructs designed to interfere with the formation of functional dopamine D2 and/or NMDA (N-methyl-D-aspartate) receptors and tested whether monkeys with the DNA treatment could learn to associate visual cues with the workload to be completed before reward delivery. The NMDA receptor was included as a target for two reasons. First, NMDA are abundant in the rhinal cortex (Kohama et al., Brain Res., 769:44-56 (1997)), and an alternative hypothesis suggests that NMDA receptors are critical for some aspects of associative learning (Bear et al., Curr. Opin. Neurobiol., 4:389-399 (1994); Morris et al., Philos. Trans. R. Soc. Lond. B. Biol. Sci., 352:1489-1503 (1997); Nicoll et al., Ann. NY Acad. Sci., 868:515-525 (1999)). Second, this alternative treatment provided the means to assess the specificity of any effects observed after treatment targeting the D2 receptor.

Construction of DNA Antisense Expression Vectors

Construction of the dopamine D2 (antisense: pIRES2D2-EGFP; sense: pcDNA3.1D2N5/hisTOPO) and NMDA (antisense: pIRES2NMDA-EGFP;) receptor expression plasmids: A thirty milligram aliquot of rhesus brain tissue (stored at −80° C.) was used for isolation of total RNA (RNeasy Mini kit, Qiagen) that was used to generate cDNA using the SMART cDNA library construction kit (Clontech). A 586 bp rhesus DRD2 receptor (D2 dopamine receptor) cDNA fragment (homologous to bp 476-1062 of African green monkey D2 receptor mRNA; Genbank Accession U18547.1) or a 360 bp rhesus NMDA receptor cDNA fragment was obtained using human or mouse specific primers, using rhesus cDNA as PCR template in a GeneAmp PCR system 9600 Perkin-Elmer thermal cycler.

The DRD2 and NMDA receptor PCR products were subcloned into the eukaryotic expression vectors pIRES2-EGFP (Clontech) or pcDNA3.1/V5/hisTOPO (Invitrogen) using standard protocols. Clones containing appropriately oriented DRD2 (pIRES2D2EGFP antisense; pcDNA3.1D2/V5/hisTOPO sense) or NMDA (pIRES2NMDA-EGFP antisense) cDNA inserts were identified by DNA sequence analysis using an ABI 377 fluorescent sequencer. Aliquots of Qiagen column purified large plasmid vector preparations were dissolved at a concentration of 10 μg/μl in artificial cerebrospinal fluid (aCSF). DOTAP (1,2-dioleoyl-3-trimethy-ammonium propane; Avanti Polar Lipids) was suspended in aCSF at concentration of 10 μg/μl and sonicated in a cup sonicator (50W) until the mixture clarified. The expression vectors (pIRES2D2-EGFP, pIRES2NMDAEGFP/pcDNA3.1D2/V5/hisTOPO or pIRES2-EGFP) were complexed with the cationic lipid DOTAP by mixing the plasmid DNA (25 μg) and DOTAP (10 μg) in aCSF and incubating at 37° C. prior to injection of 1 μl aliquots into the rhesus rhinal cortex or basal ganglia as described herein.

Injection of DNA Antisense Expression Vectors

The experimental approach used here required making a series of injections, 2-3 mm apart, across the entire rhinal cortex (Baxter et al., Eur. J. Neurosci., 13:1228-1238 (2001)). After four weeks of testing, each monkey received a set of injections to introduce one of the following four agents into the rhinal cortex of the intact hemisphere: 1) Antisense DNAs targeting dopamine D2 and NMDA receptors (n=2 monkeys); 2) Antisense DNA targeting only the D2 receptor (n=2); 3) Antisense DNA targeting the NMDA receptor (n=2); or 4) vector only (n=1). For the rhinal cortical injections, DNA suspension was injected under direct visualization using a surgical microscope. Each 1.0 μl injection was made into cortex along and on each side of the rostral-caudal extent of the rhinal sulcus via the 30-gauge needle of a 10 μl Hamilton syringe. Sites were placed approximately 2 mm apart and were intended to include all of areas 28, 35, and 36, similar to that reported previously (Tang et al., supra). The number of injection sites was 39±1 (mean±SEM, n=11).

Following the injections, monkeys were given 3 weeks to recover. In all cases recovery was uneventful. The testing procedure after the recovery period was as follows: monkeys were trained with the schedules using a new set of cues (length cues; FIG. 1c) for three consecutive weeks, followed by one week of a cue discrimination testing (as a control), and then, at least one additional week (4^{th} week) of training with the schedules using the length cues. After this initial testing, the monkeys were rested for two weeks and tested for two weeks in alternation until they learned.

By the second week, the performance of the 3 monkeys receiving either the NMDA receptor targeted treatment or vector only treatment was the same as before the treatment. That is, the relationship between the error rates and schedule states obtained with the new length cues was statistically indistinguishable from the relationship observed before treatment (interaction term between schedule states and week of testing in a two way ANOVA, F_{5,35}=1.65, p=0.18). The behavior was stable from the 2^{nd} to the 4^{th} week (interaction term of a two way ANOVA, F_{10,53}=0.287, p=0.98). Thus, monkeys receiving treatment targeting rhinal cortex NMDA receptors or vector only learned new cue sets at a rate similar to that measured before the treatment, indicating that these treatments were without effect.

All four monkeys receiving D2 receptor targeted treatment (two monkeys for combined D2 and NMDA receptors and two for D2 receptor alone) failed to adjust their error rates across different schedule states for 11 to 19 weeks after the injections (Table 1; FIG. 3b, c). During the 11-19 weeks, monkeys receiving DNA constructs targeting the D2 receptor showed the same deficit in associating visual cues with reward schedules as observed in monkeys with bilateral rhinal cortex removals (Liu et al., supra). After regaining the ability to use the cues, the behavior was stable; the relationships between the average error rates and schedule states were the same from the 1^{st} to the 3^{rd} week after cues were learned (interaction term between schedule states and week of testing in a two way ANOVA, F_{10.71}=0.367, p=0.96). This suggests that the effect of this DNA treatment is temporary.

To further demonstrate that the DNA treatment targeting D2 receptor was responsible for these reversible behavioral alterations, 4 of the 7 previously injected monkeys were given repeat injections of the combination of D2 and NMDA receptor constructs (n=1) or D2 receptor constructs alone (n=3). All four of those monkeys showed another prolonged period during which a new set of visual cues (pattern cues-2; FIG. 1c) failed to guide their behavior (FIG. 3d). As before, all 4 of these animals learned this cue set a minimum of 11 weeks after the injections (Table 1). Subsequently, all 4 monkeys learned a 5th cue set (pattern cues-3; FIG. 1c) during the first week after it was introduced. The reinstatement of the learning deficit after the 2^{nd} treatment adds weight to the argument given earlier, that the recovery from the effect of the 1^{st} treatment was due to restoration of D2 receptors in the rhinal cortex, as opposed to compensatory contribution of other brain regions.

These results indicate that each of the 8 instances involving injections of DNA targeting D2 receptor (D2 receptor alone: 4 monkeys with a total 5 treatments; D2+NMDA receptors: 3 monkeys with 1 treatment each) caused severe impairments in learning associations between visual cues and predicting workload before reward.

TABLE 1
Treatment Summary.

| | Side of | Side of | Initial Treatment | | | Repeat Treatment | | |
|---|---|---|---|---|---|---|---|---|
| | Rhinal | Rhinal | | Number | | | Number | |
| Monkey | Cortex | Cortex | Antisense DNA | of | Impairment | Antisense DNA | of | Impairment |
| Number | Removal | Injection | Construct | Injections | (weeks) | Construct | Injections | (weeks) |
| 1 | Right | Left | D2 + NMDA | 41 | yes (15) | — | — | — |
| 2 | Right | Left | D2 + NMDA | 43 | yes (11) | — | — | — |
| 7 | Left | Right | D2 | 37 | yes (12) | — | — | — |
| 5 | Left | Right | D2 | 38 | yes (19) | D2 | 39 | yes (11) |
| 4 | Left | Right | NMDA | 44 | no | D2 | 36 | yes (12) |
| 6 | Left | Right | NMDA | 35 | no | D2 | 39 | yes (11) |
| 3 | Left | Right | Vector | 39 | no | D2 + NMDA | 44 | yes (11) |

The “Right” and “Left” in the second and third columns indicate the hemisphere in which the operation was performed.
“D2” and “NMDA” indicate DNA constructs targeting D2 or NMDA receptor proteins, respectively.
“Vector” indicates an empty vector without specific DNA.
The numerals in parentheses after “yes” indicate the number of weeks the monkey displayed the impairment.

### Example 3

### Effect of Antisense Oligonucleotide Administration on Relearning

To determine whether the relearning was due to many weeks of practice with a specific cue set, as opposed to recovery from treatment, the 4 monkeys were presented with another new cue set (pattern cues-1; FIG. 1c). After 2 weeks of practice with the pattern cues, the relationship between error rates and schedule states was indistinguishable from that observed using the initial cue set (brightness cues), prior to any injections (interaction term of a two-way ANOVA, F_{5,47}=0.736, p=0.60; FIG. 3c).

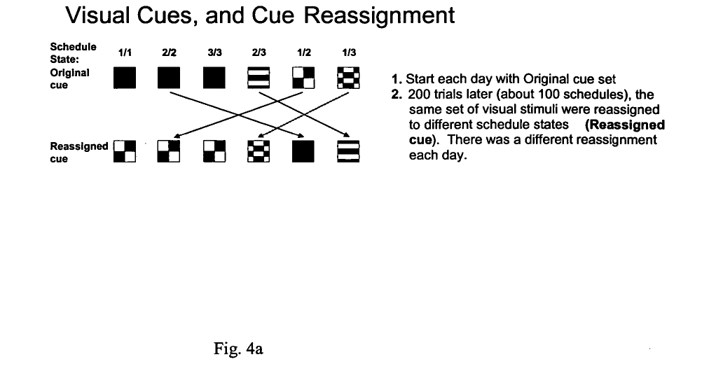

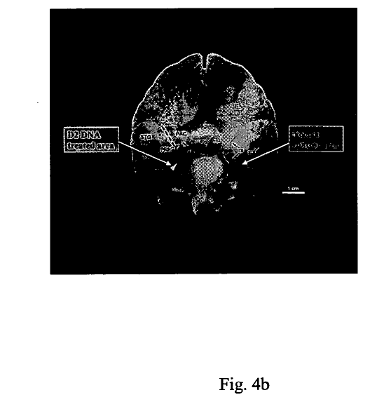

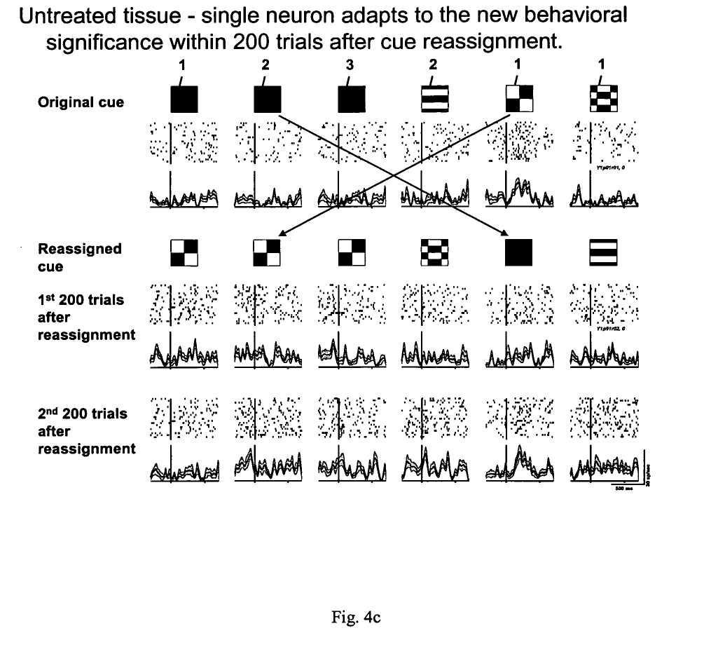

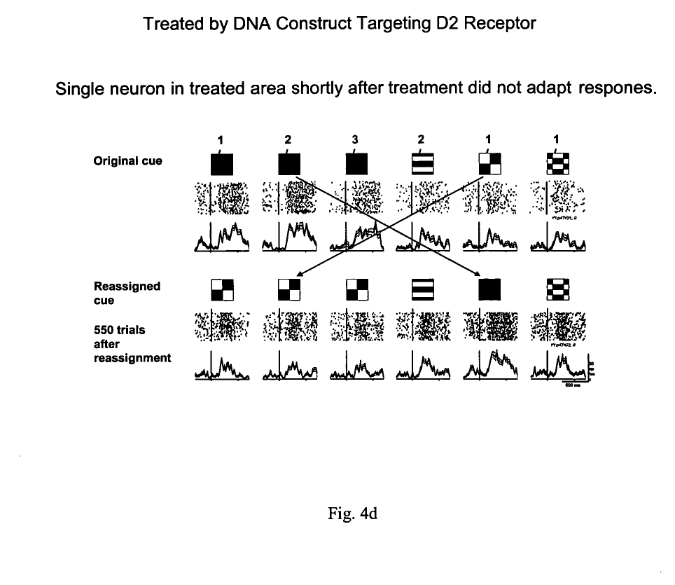

To further analyze the effect of DNA treatment on learning, single neuron recording was performed on neurons in normal (untreated) and in DNA treated rhinal cortices of monkeys. As shown in FIG. 4a, visual cues were reassigned. Each day, a monkey was tested with an original cue set. Two hundred trials later (approximately 100 schedules), the same set of visual stimuli were reassigned to different schedule states (FIG. 4a, “Reassigned cue”). The visual stimuli were reassigned every day.

Single neuron recording was performed following unilateral DNA treatment. DNA was injected into a small cube (3 mm³) of perirhinal cortex in one hemisphere (FIG. 4b, “D2 DNA treated area”). Single neurons from perirhinal cortex were recorded between three and nine weeks after the injections, from both DNA treated and normal control areas. (Recording sites were localized using MRI.) Single neuron recordings were again performed 18 weeks after the injections. 108 perirhinal neurons from two monkeys were recorded (35 from normal control area, 38 from treated area after DNA injections, and 35 from treated area 18 weeks after treatment).

As shown in FIG. 4c, single neurons from normal control area adapt to the reassigned visual cues within 200 trials after cue reassignment. Single neurons from DNA treated area, however, did not adapt to the reassigned visual cues (FIG. 4d).

This finding, that the ability to learn new cues recovered after treatment and proceeded at the same rate as before DNA treatment, strongly suggests that the D2 receptor targeted DNA treatment had a time-limited, reversible effect on cognitive behavior, similar to what Davidkova et al. reported using this approach in mice (J. Pharmacol. Exp. Ther., 285:1187-1196 (1998)). Given the normal learning profile, an alternative explanation, that another brain region mediated acquisition of the new set of visual cues, is unlikely.

### Example 4

### Effect of Antisense Oligonucleotide Administration on Cue Discrimination

Finally, to determine whether monkeys receiving D2 receptor targeted treatment could distinguish among the length cues, a control procedure was carried out wherein the length cues were used in place of the red and green spots in the visual discrimination trials (Post-injection testing: Monkeys were given 3 weeks to recover from the injections. In all cases recovery was uneventful. The testing procedure after the recovery period was as follows: monkeys were trained with the schedules using the length cues for three consecutive weeks, followed by one week of a cue discrimination testing (as a control), and then at least one additional week (4^{th} week) of training with the schedules using the length cues. After this initial testing, the monkeys were rested for two weeks and tested for two weeks in alternation until they learned). Using the two cues with smallest length difference in place of the red and green spots, the monkeys immediately performed the cue discrimination task with better than 90% correct responses. These results indicate that during the period in which the monkeys were impaired on the reward schedule task, they could easily discriminate the rectangles in the length cue set.

### Example 5

### Effect of Antisense Oligonucleotide Administration on D2 Receptor Levels

To test whether our DNA treatments were specific to the targeted receptor, receptor binding studies were conducted using two experimentally naïve rhesus monkeys. D2 receptor targeted DNA was injected unilaterally into the rhinal cortex of a monkey that was sacrificed 7 weeks after treatment. Similarly, NMDA receptor targeted DNA was injected unilaterally into the rhinal cortex of the other monkey, which was sacrificed 3 weeks following treatment. Both time points for sacrifice were within the time period that behavioral effects were evident after D2 receptor DNA injection. Histological sections were prepared using [¹²⁵I]-Iodosulpride (Amersham Biosciences, Piscataway, N.J.) and [³H]-MK-801 (American Radiochemicals Inc, St. Louis, Mo.) to label D2 receptors and NMDA receptors, respectively. After exposure and development of the autoradiographs, multiple measurements were made at matched sites throughout the rhinal cortices of both hemispheres (Bouthenet et al., Neuroscience, 20:117-155 (1987); Ibrahim et al., Molecular Brain Res., 79:1-17 (2000)).

Radioligand receptor autoradiography was performed on slide-mounted 14 μm coronal sections throughout the rostral-caudal levels of the rhinal cortex to label D2 and NMDA receptors. [¹²⁵I]-Iodosulpride (Amersham Biosciences, Piscataway, N.J.) was used to label D2 receptors as described by Bouthenet et al. (supra). Sections were allowed to thaw and were preincubated in buffer (50 mM Tris HCl; 120 mM NaCl; 5 mM KCl; 1 mM CaCl₂; 1 mM MgCl₂; and 5.7 mM ascorbic acid) at room temperature, RT, for 10 min. Slides were then incubated in buffer containing 3.27 nM [¹²⁵I]-Iodosulpride for 30 minutes. The sections were then rinsed twice for 5 min each in ice-cold buffer and dipped briefly in ice-cold distilled water. Two to three adjacent brain sections were incubated in the presence of 1 μM domperidone to determine non-specific binding. [³H]-MK-801 (American Radiochemicals Inc, St. Louis, Mo.) was used to label NMDA receptors as described by Ibrahim et al. (supra). Sections were allowed to thaw, were pre-incubated in ice-cold buffer (5 mM Tris HCl; 50 μM L-glutamate; 50 μM spearmine; and 50 μM glycine) for 20 min, dipped briefly in distilled water then allowed to dry. Once dry, the slides were incubated in buffer containing 3.60 nM [³H]-MK-801 at RT. The sections were then washed three times for 5 minutes in ice-cold buffer and briefly dipped in ice-cold distilled water. Two adjacent brain sections were incubated in the presence of 10 μM MK-801 to determine non-specific binding. All slides were allowed to dry under a stream of air and were apposed to Kodak Biomax MR film with polymer-calibrated [¹²⁵I]- or [³H] microscale standards, (Amersham Biosciences, Piscataway, N.J.) at RT (10 day exposure for [¹²⁵I]-Iodosulpride and four week exposure for [³H]-MK-801).

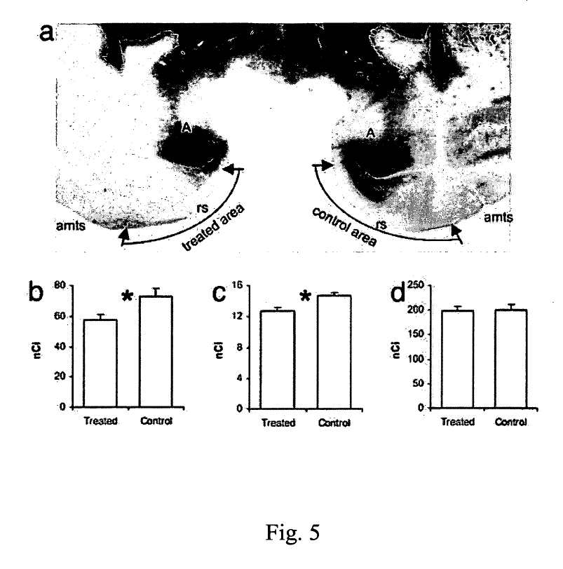

After exposure and development of the autoradiographs, multiple measurements were made at matched sites throughout the rhinal cortices of both hemispheres. The injection sites could be identified under low-power magnification as discrete needle penetration tracks with small amounts of localized gliosis surrounding the injection sites. Optical densities of the receptor expression on the film images were quantified using NIH Image (available on the internet at rsb.info.nih.gov/ij). Four sections approximately equally spaced through the rhinal cortex were selected. The ROI's shown in the representative sections in FIG. 5a were selected and lines were run through the section at 12 locations per hemisphere spread across the rhinal cortex. The locations in the two hemispheres were matched according to anterior-posterior location and intrasectional landmarks, e.g., distance from the rhinal sulcus. The peak optical density along each line was taken as the measure for that location. 48 measurements from each hemisphere were taken for each receptor binding study.

As expected, autoradiographs of the D2 receptor DNA treated brain showed significant decreases in D2 receptor density in the rhinal cortex of the treated hemisphere compared with the untreated (control) hemisphere (FIG. 5a, b; D2 receptor treated side, 57.3±3.5 nCi, mean±sem (n=36 measurements/side); control side, 73.1+5.0 nCi; paired t-test, t47=−3.5, p=0.0005). Our results with D2 receptor targeted treatment are consistent with the findings using a similar technique in mice (Weiss et al., Neurochem. Int., 31:571-580 (1997); Weiss et al., Cell Mol. Life Sci., 55:334-358 (1999)), and show that manipulation of the D2 receptor is a viable explanation of the deficit that occurred after injection of DNA.

In the brain treated with NMDA receptor targeted DNA, there was a significant decrease in MK801 binding in the rhinal cortex on the NMDA receptor DNA treated side relative to the untreated side (FIG. 5c; NMDA receptor treated side, 12.8+0.4 nCi; control side, 14.7+0.4 nCi; paired t-test, t47=−3.1, p=0.0015). Thus, although the NMDA receptor DNA treatment did significantly decrease the density of NMDA receptors, there was no effect of this treatment on learning associations between visual cues and predicted workload. In addition, the NMDA receptor DNA treatment did not alter the density of D2 receptors (FIG. 5d; D2 receptor binding of NMDA receptor DNA treated side, 197.9±10.6 nCi; D2 receptor binding of control side, 200.2±10.2 nCi; paired t-test, t47=−0.5, p=0.299).

These results indicate that the D2 and NMDA receptor targeting constructs significantly decrease the ligand binding of the intended receptors, thereby supporting the conclusion that the D2 receptor manipulation was responsible for the cognitive deficit.

### Example 6

### Making a Reversible Monkey Model of Hemi-Parkinsonism

Targeting the D2 Dopamine Receptor

A monkey model of hemi-parkinsonism was made by unilaterally administering the D2 DNA antisense expression vector described in Example 2 into the head of the caudate and the putamen of the basal ganglia. The experimental approach used here required making a series of injections, 2-3 mm apart, across the entire head of the caudate and the putamen. For the injections, DNA suspension was injected under direct visualization using a surgical microscope. Each 1.0 μl injection was made using the 30-gauge needle of a 10 μl Hamilton syringe. Sites were placed approximately 2 mm apart. The number of injection sites was 35 in the head of the caudate and 50 in the putamen.

Following a 2-day recovery period, the monkey was observed for any motor defects. The monkey exhibited delayed initiation of reaching using the limb that was contralateral to the injected side of the basal ganglia. Further, the monkey also exhibited decomposition using the contralateral limb. These motor defects were temporary, and were no longer observed 4-6 weeks following the injections.

Targeting Tyrosine Hydroxylase

A monkey model of hemi-parkinsonism is made by administering a nucleic acid construct encoding an antisense oligonucleotide that targets a gene encoding tyrosine hydroxylase. The nucleic acid construct is injected unilaterally within the basal ganglia region of the brain using a 30 gauge 10 μl Hamilton syringe. A nucleic acid construct that does not target a gene encoding tyrosine hydroxylase (vector alone) is injected unilaterally into the basal ganglia of the opposite side of the brain. The treated monkey is allowed to recover from the administration, i.e., until the activity of the untreated side regains normal function. The induction of Parkinsonism within the antisense oligonucleotide-treated side of the brain is evaluated by observing the clinical motor deficits, e.g., tremor or trembling in hands, arms, legs, jaw, and face; rigidity or stiffness of the limbs and trunk; bradykinesia; and postural instability or impaired balance and coordination. The monkey is also evaluated for the ability to perform a learning memory task. The monkey is evaluated daily to determine the time for Parkinsonism symptoms induced in the antisense oligonucleotide-treated side of the brain to attenuate. Following attenuation, the nucleic acid construct encoding the antisense oligonucleotide is administered to the opposite side of the brain (that was previously treated with vector alone), and the monkey is reevaluated.

The hemi-parkinsonism monkey model is used to test the efficacy of compounds such as L-DOPA in treating the symptoms by comparing the effects of L-DOPA on the activity of the antisense oligonucleotide-treated side of the brain to the effects of L-DOPA on the activity of the normal (vector alone-treated) side of the brain. Other experimental compounds are tested in the same manner.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of making an animal model of a neurological disorder, the method comprising administering to a brain region of the animal a nucleic acid construct comprising a nucleotide sequence that is complementary to a portion of an mRNA of a target gene encoding a protein the activity of which is associated with the disorder, in an amount effective to inhibit translation of the mRNA, thereby inducing the disorder in the animal.

2. The method of claim 1, wherein the animal is a non-human primate.

3. The method of claim 1, wherein the nucleic acid construct is administered to one side of the brain.

4. The method of claim 1, wherein the disorder is schizophrenia.

5. The method of claim 1, wherein the disorder is cocaine use/abuse.

6. The method of claim 1, wherein the disorder is Parkinson's disease.

7. The method of claim 6, wherein the brain region is the basal ganglia.

8. The method of claim 6, wherein the target gene encodes a protein within the dopamine pathway.

9. The method of claim 1, wherein the target gene encodes tyrosine hydroxylase.

10. An animal model of a neurological disorder produced by administering to a brain region of the animal a nucleic acid construct comprising a nucleotide sequence that is complementary to a portion of an mRNA of a target gene encoding a protein the activity of which is associated with the disorder, in an amount effective to inhibit translation of the mRNA, thereby inducing the disorder in the animal.

11. An animal model of a neurological disorder caused by decreased expression of a target gene, wherein the animal has inserted into a region of its brain a nucleic acid construct comprising a nucleotide sequence that is complementary to a portion of an mRNA of the target gene, in an amount effective to inhibit translation of the mRNA.

12. The method of claim 11, wherein the gene is the D2 dopamine receptor and the disorder is selected from the group consisting of Parkinson's Disease, obsessive compulsive disorder, schizophrenia, and drug abuse.

13. The method of claim 11, wherein the gene is dopamine β-hydroxylase and the disorder is affective disorder.

14. The method of claim 11, wherein the gene is selected from the group consisting of neuregulin 1, dystrobrevin binding protein, and disrupted in schizophrenia-1 (DISC-1), and the disorder is schizophrenia.

15. The method of claim 11, wherein the animal is a non-human primate.

16. The method of claim 11, wherein the nucleic acid construct is administered only to one hemisphere of the brain.

17. The method of claim 16, wherein the hemisphere to which the nucleic acid construct is not administered is a reference.

18. The method of claim 11, wherein the nucleic acid construct comprises DNA.

19. The method of claim 11, wherein the nucleic acid comprises an expression vector.

20. The method of claim 11, wherein the induction of the disorder is not permanent.

21. A method of testing a potential therapy for treatment of a neurological disorder caused by or associated with increased activity of a protein encoded by a gene, the method comprising:
administering to a brain region of a test subject not having the disorder a nucleic acid construct comprising a nucleotide sequence that is complementary to a portion of an mRNA of the gene in an amount effective to inhibit translation of the mRNA, thereby inducing the disorder in the test subject;
administering a potential therapy; and
evaluating an effect of the potential therapy on a clinical parameter of the disorder, wherein an improvement in the clinical parameter indicates that the therapy is effective in treating the neurological disorder.

22. The method of claim 21, wherein the gene is the D2 dopamine receptor and the disorder is selected from the group consisting of Parkinson's Disease, obsessive compulsive disorder, schizophrenia, and drug abuse.

23. The method of claim 21, wherein the gene is dopamine β-hydroxylase and the disorder is affective disorder.

24. The method of claim 21, wherein the gene is selected from the group consisting of neuregulin 1, dystrobrevin binding protein, and disrupted in schizophrenia-1 (DISC-1), and the disorder is schizophrenia.

25. The method of claim 21, wherein the test subject is an animal.

26. The method of claim 25, wherein the animal is a non-human primate.

27. The method of claim 21, wherein the improvement is relative to a control or reference subject.

28. The method of claim 27, wherein the reference subject is a subject that has not been administered the therapy.

29. The method of claim 27, wherein the reference subject is the same individual as the test subject, prior to induction of the disorder.

30. The method of claim 21, wherein the nucleic acid construct is administered only to one hemisphere of the brain.

31. The method of claim 30, wherein the hemisphere to which the nucleic acid construct is not administered is a reference.

32. The method of claim 21, wherein the nucleic acid construct comprises DNA.

33. The method of claim 21, wherein the nucleic acid comprises an expression vector.

34. The method of claim 21, wherein the portion of the mRNA is within the translated region.

35. The method of claim 21, wherein the therapy comprises administering a test compound.

36. The method of claim 35, wherein the test compound is selected from the group consisting of small organic or inorganic molecules, peptides, polypeptides, nucleic acid sequences, and polysaccharides.

37. The method of claim 21, wherein the therapy is a permanent treatment.

38. The method of claim 37, wherein the permanent treatment is surgery.

39. The method of claim 21, wherein the therapy is administration of a noncompetitive inhibitor.

40. The method of claim 21, wherein the therapy is the administration of a viral vector.

41. A method of selecting a candidate nucleic acid construct for the treatment of a neurological disorder, the method comprising:
administering to a brain region of a subject having the disorder a test nucleic acid construct comprising a nucleotide sequence that is complementary to a portion of a mRNA strand of a pre-selected target gene in an amount effective to inhibit translation of the mRNA in a control subject;
evaluating an effect on a clinical parameter of the disorder; and
wherein a test nucleic acid construct that provides a positive effect on a clinical parameter is a potential treatment of the disorder.

42. The method of claim 41, wherein the effect is evaluated compared to a control or reference.

43. The method of claim 41, wherein the gene is the D2 dopamine receptor and the disorder is selected from the group consisting of Parkinson's Disease, obsessive compulsive disorder, schizophrenia, and drug abuse.

44. The method of claim 41, wherein the gene is dopamine β-hydroxylase and the disorder is affective disorder.

45. The method of claim 41, wherein the gene is selected from the group consisting of neuregulin 1, dystrobrevin binding protein, and disrupted in schizophrenia-1 a (DISC-1), and the disorder is schizophrenia.

46. The method of claim 41, wherein the nucleic acid construct comprises DNA.

47. The method of claim 41, wherein the nucleic acid comprises an expression vector.

48. The method of claim 41, wherein the subject is an animal.

49. The method of claim 48, wherein the animal is a non-human primate.

50. The method of claim 48, wherein the animal is a human.

51. The method of claim 41, wherein the improvement is relative to a control or reference subject.

52. The method of claim 51, wherein the reference subject is a subject that has not been administered the therapy.

53. The method of claim 51, wherein the reference subject is the same subject, prior to induction of the disorder.

54. The method of claim 41, wherein the nucleic acid construct is administered only to one hemisphere of the brain.

55. The method of claim 54, wherein the hemisphere to which the nucleic acid construct is not administered is a reference.
